SEQUENCE LISTING (4 sequences)

SEQ ID NO: 1 - DNA, 1096 bp, Uromyces fabae
  PMA1 ATPase promoter AJ003067.1
gcgcagaattttgaaacttgagattgtagcagttgtgccaagagaatcgatctgaaagtatgtatataaagtgagataactacataaacctggttaagcgttgcagtgccaaagcgaggaagagtcaagacgaagagtaattgcattacgtaatgccgaccccctacggcaggggttatgtactaagaagtcagcactataccgtagtacgcactcagacaggctgggcagtagtttttgtacagaagctgaggtctgggcactcatgaattctcccgcgaaaccaaggtccaatgtggagcgaggtcctcgaagagttgatcgactggcagaagcttggcatgtctatggggtgtatatatagcaatgcaatgtactccccctcagcgttcaatatctcaaacctcccctgacacacatcgtcaactgcagtattatttatctcgcgagtttgtccattccccatccgttgtcaccctgttcagtcccgagtttgatacgattaaaaggaagatcagagccatcggggtgtcggaagttgtactagtgtcttctattgctaaacttgttcttttttcatttttattttccgattagccatatatatcatcgcagttggttggatatttaaactagcgcttccttcgcccatcacttcttcttccttccttgtgttggtaggtgttgtggcccgtggacccgtggtgttcctttcctcaataatttattcacacactctcctcaacccagaccccctgactaacatcttcatatcgtcggctctgactaccatagcgcatgagactaggctattgaagcatttagttcccaccctgtcacatcaccgtcgccctcacaaccttgtccttcagttgcactttagttgcccttgagcctccatccgcccaccacctataaatcctcccaccaaactttctcagcccccggagccctttggatccactcttctataccgcctagttttatttcgtcttatactattattttacctgcttaatttttcattttttacttttctttacctgccacaacctactctctagctttaatcatctcttaattactaatcagaatg

SEQ ID NO: 2 - DNA, 619 bp, Uromyces fabae
  PMA1 ATPase terminator AJ003067.1
ctacttccccagatgtccatgcttttgggcggtttgtactttttgtcattatcctttacatctacgtgttatctaatagctgcgacaattcaatagctgaagcatatcacactgcgtagtaacgcgcagggcctgtaaatattccgtttgaattcatgcacatacaaacagacctttttttcgttttacttgtctttcgttcggcgttgtcatcggttgcgggtttccttgttactatcctctctttttttcgccttttgcaacatttcttcttctttcattttcattcttctttgtcctatctatttcacttgaacctctggtctgattacagttagggtcacatacccaggcagattgaattattgttcaatgacaaaacatcaacagctaggttaactgccattaaagtagcttgttgaaatagaactttaagcttcctctttcattagaaataattaataatatatgcattatctcatttcatttgatgcctgcttcattagcaaattcatctcttaccttctgtgccatttgctcctctttttcattagaatgccagtttcatcgggcaattcatatctttctgagacaatattgtgactccaggctgtagcga

SEQ ID NO: 3 - protein, 283 aa, Uromyces fabae
  SucDH1(H254Y) protein
MINIPNSIRPFIRSANRTPCYLRSISSSSSSSFATPAEEHAGKQPSSAVPVKEFSIYRWNPDEPSKKPTLQTYSIDLKKCGPMVLDALIKIKNELDPTLTFRRSCREGICGSCAMNIDGVNTLACLKRINKETSAPVKIYPLPHMYIIKDLVPDMTHFYKQYKSIEPFLKNDNPPAQGEFLQSPEDRKKLDGMYECILCACCSTSCPSYWWNQDEYLGPAVLMQAYRWMADSRDSYGEDRKEKLQNTLSLYRCYTIFNCTKTCPKGLNPAKAISHIKREMASA

SEQ ID NO: 4 - DNA, 1240 bp, Uromyces fabae
  SucDH1(H254Y) mRNA given as DNA
atgatcaacattccgaattcgatcagacctttcatacgatcagctaatcgcaccgtaagcatccattcactccttatttgctctgtttttgtcaaccaaagaacttttcaactcacttgatttcatcttgttgttttttaaagccatgctatttgcgctcgatatcatcaagtagctcatcctcttttgccaccccagcagaagaacatgctggtaaacagccatcttcagcggtgcctgtcaaagagttttccatttaccgttggaatccggatgaaccgagtaagaagccgacgttacaaacctattcaattgacttaaaaaaatgcggacccatggttcttgatgccctaatcaagatcaagaacgaactcgatccaaccttgacctttcgtagaagttgtcgagaaggtatctgtggtagctgtgccatgaatattgatggtgtcaacacgctggcttgtttaaagcgcatcaacaaagaaactagtgctccggtcaaaatctaccctctccctcatagtatgtctgcctcacctaaaatataaatttcatctcttgatctcattttttctaaaacgaccagtgtacataattaaggatttagttccagatatgacccacttctacaagcagtacaagtcgattgaacctttcttaaagaacgataatcccccagctcaaggtgaattccttcagtctcctgaagaccgcaagaaactcgatgggatgtacgagtgcatcctttgtgcatgttgctgttcgtatacctttatctcttggttttcaatttaatcacaagcctaaccgaactttatttaacctgacattcttagccactagttgtcccagttactggtggaatcaagatgaatatttgggtccggctgtgctaatgcaagcttatcggtatgtccattttcaattccattgcattgtaactttttgtctcactgaacgcaattcattttctctcttgttcaatgtagatggatggccgactcgcgggattcatatggagaggaccgaaaagagaaacttcagaacacgctctctctctacagatgttacacaattttcaattgcaccaaaacctgtccgaaaggtttgaacgtgagaggttttttttttactcaagtctacattcagctttcactgattcaattgggttgtttcatctctttctttcaagccggcaaaagccatatcgcacattaaaagggagatggcatcagcctga

(19) United States
(12) Patent Application Publication
(10) Pub. No.: US 2022/0340918 A1
(43) Pub. Date: Oct. 27, 2022

(54) METHODS FOR TRANSFORMATION OF FUNGAL SPORES

(71) Applicant: BASF PLANT SCIENCE COMPANY GMBH, Ludwigshafen (DE)

(72) Inventors: Maarten Hendrik Stuiver, Zwijnaarde (BE); Doreen Schachtschabel, Ludwigshafen (DE); Birgit Hoff, Ludwigshafen (DE); Tobias Mentzel, Mannheim (DE); Isabella Siepe, Limbrrgerhof (DE); Eckhard Thines, Kaiserslautern (DE); Stefan Jacob, Kaiserslautern (DE); Michael Becker, Kaiserslautern (DE); Alexander Yemelin, Kaiserslautern (DE)

(21) Appl. No.: 17/620,775

(22) PCT Filed: Jun. 17, 2020
(86) PCT No.: PCT/EP2020/066828

§ 371 Date: Dec. 20, 2021

(30) Foreign Application Priority Data
Jun. 27, 2019 (EP) 19182791.4
Dec. 18, 2019 (EP) 19217396.1

(51) Int. Cl. C12N 15/82; C12N 15/87; C12N 15/80

(52) U.S. Cl. CPC C12N 15/8206; C12N 15/87; C12N 15/80

(57) ABSTRACT

The present invention provides to a method for the introducing polynucleotide molecules into fungal spores comprising exposing a mixture of fungal spores and magnetic nanoparticles carrying the polynucleotide to a magnet and/or a magnetic field. The present invention also provides a method for transformation a fungus comprising the steps of the method steps disclosed herein and allowing the integration of said polynucleotide molecules into the genome of said spores, thereby transforming the spore. The present invention also provides a system for delivery of nucleic acids to fungal spores comprising magnetic nanoparticles loaded with nucleic acids, a kit for transformation of fungal spores with a polynucleotide comprising MNPs loaded with the polynucleotide and fungal spores as well as a composition comprising MNPs loaded with nucleic acid molecules and fungal spores.

### FIELD OF THE INVENTION

The invention is directed to transformation of fungal spores.

### DESCRIPTION OF THE INVENTION

Fungi are excellent cell factories and find a wide application in the production of many useful complex chemical compounds. They can grow rapidly in simple and inexpensive media. Genetic transformation technologies allow the genetical modification of fungal genome. As eukaryotes fungi contain larger genomes, can therefore be transformed with large constructs and their products are often more applicable for human use than products from bacteria. The transformation of fungi allows not only to investigate the fungal metabolism but allows the insertion of new genetic elements and the modification of endogenous genes. Thus, the transformation of fungi is a key step in any development of new fungal strains for biotechnological methods. Transformation of fungi shall be possible for all fungal species at least grown in culture (Fincham, Microbiol Rev. 1989 March; 53(1): 148-170.

Many general methods of genetic transformation for fungi, including protoplast-mediated transformation, Agrobacterium-mediated transformation, electroporation, biolistic method and shock-wave-mediated transformation are known (Li, D., Tang, Y., Lin, J. et al. Microb Cell Fact 16, 168 (2017))

Thus, though many transformation methods of fungi are known in the art, a number of fungi are recalcitrant to be transformed with these methods and the methods are often time consuming and not very efficient.

In view of the foregoing, the present invention provides new compositions and new methods for the transformation of fungal spores.

Consequently, the present invention provides to a method for the introducing polynucleotide molecules into fungal spores comprising exposing a mixture of fungal spores and magnetic nanoparticles carrying the polynucleotide to a magnet and/or a magnetic field.

Further, the present invention provides a method for introducing polynucleotide molecules into fungal spores comprising the steps of:
a. loading the polynucleotide molecules to be transferred into the spore on magnetic nanoparticles (MNP), and
b. adding said polynucleotide molecules loaded on said magnetic nanoparticles (DNA-MNP) to said spores, and
c. exposing the mixture to a magnetic force, e.g. a magnet and/or a magnetic field,
d. incubating the mixture in presence of said magnet to allow introduction of the polynucleotide molecules into said spores,
   thereby introducing the polynucleotide molecules into said spores.

The present invention also provides a method for transformation a fungus comprising the steps of the method steps disclosed herein and allowing the integration of said polynucleotide molecules into the genome of said spores, thereby transforming the spore.

The present invention also provides a system for delivery of nucleic acids to fungal spores comprising magnetic nanoparticles loaded with nucleic acids, as well as a kit for transformation of fungal spores with a polynucleotide comprising MNPs loaded with the polynucleotide, and fungal spores, as well as a composition comprising MNPs loaded with nucleic acid molecules, and fungal spores.

### DETAILED DESCRIPTION OF THE INVENTION

The delivery of genetic materials into target cells is a powerful tool to manipulate or modify the activities of nucleic acids in the cells. One well-established method for the delivery of introducing DNA in target cells is electroporation. As summarized in US2008075701 the electroporation method has two main fatal disadvantages: low delivery rate and low cell survival rate.

A relatively new method for delivering genetic materials into target cells is magnetofection. For a magnetofection, the genetic materials are loaded on magnetic nano particles coated with cationic molecules (MNP). The magnetic particles-genetic materials complexes are then transported into cells under the influence of an external magnetic field. Many reports have demonstrated that magnetofection is nontoxic, highly efficient and versatile (US2008075701).

In magnetofection, conjugated magnetic nanoparticles (MNPs) are bound to a target molecule, such as nucleic acid, and a magnetic field then is applied to the molecule-bound MNPs to deliberately introduce and concentrate the particles into one or more target cells. The nucleic acids can then be released into the cell cytoplasm by various different mechanisms.

Fungi have complex life cycles and transformation method protocols have to be adapted for each new species, e.g. to their specific life cycle phases, growth conditions, cell wall composition, environment etc. For examples even for the different stages of one species (like S. cerevisiae) distinct transformation protocols have been developed. So, dependent on the fungal species genetic material is delivered into for example, whole cells, conidiospores, conidia, spores, protoplasts, myceliums, etc by methods like protoplast-mediated transformation, Agrobacterium-mediated transformation, electroporation, biolistic method and shockwave-mediated transformation. The efficient is often very low.

Delivery of DNA to fungal spores has not been widely reported.

It has been now be found, that the use of magnetic nanoparticles (MNPs) coated with positively charged polymers is applicable to fungal spores and provides an efficient and rapid method for the introduction of polynucleotides into fungi spores of very distinct fungal species.

Deliver the genetic material to fungal spores is more efficient and much quicker than the existing methods. The method allows the delivery of nucleic acids to fungal species to which until now polynucleotides could not be delivered or only after difficult and time-consuming transition to other stages of the fungal life cycle, e.g. to protoplasts. For example, some fungi that are obligate plant pathogens require plant hosts for their growth and amplification and can only be isolated as spores from said host plants. Modification of these fungi is difficult if not impossible. In addition, spores are easy to harvest, store and may withstand rigid conditions, all facilitating the handling of the biological material in many situations.

Thus, the present invention provides a method for the introducing polynucleotide molecules into fungal spores comprising exposing a mixture of fungal spores and magnetic nanoparticles carrying the polynucleotide to a magnet and/or a magnetic field.

In one embodiment, the method of the present invention comprises the following steps:
a. for example, providing fungal spores,
b. loading the polynucleotide molecules to be transferred into the spore on magnetic nanoparticles (MNP), and
c. adding said polynucleotide molecules loaded on said magnetic nanoparticles (DNA-MNP) to said spores, and
d. exposing the mixture to a magnetic force, e.g. a magnet and/or a magnetic field,
e. incubating the mixture in presence of said magnet to allow introduction of the polynucleotide molecules into said spores,
   thereby introducing the polynucleotide molecules into said spores.

As shown herein, magnetofection can be used as highly efficient gene delivery method for a wide spectrum of fungal spores.

Recently a method for transformation of plant pollen has been reported using magnetic nanoparticles coated with polyethyleneimine (Zhao et al, 2017, nature plants 3, 956-964).

Plant pollen comprises an inner and an outer wall. The outer wall contains pores. The pollen can swell and shrink resulting from the moisture within the pollen. The pores' outer wall allows the uptake of the moisture. The inner wall consists of cellulose and hemicellulose, and contains callose as well. The outer wall consists mainly of sporopollenin, a mixture of various biopolymers including mainly long chain fatty adds, phenylpropanoids, phenolics, carotenoids and xanthophyll. The outer coat of the plant pollen is made of proteins and is called exine. Zhao et al indicates that having pores in the outer wall is an important determinant for the ability of nano-magnets to transform plant pollen. Cotton pollen, as used by Zhao et al., have pores in the exine (outer) cell wall of about 5-10 □m.

In contrast, the composition and structure of the fungal cell wall is completely different than that of plant pollen. The fungal spore cell is very rigid and able to contain a high intracellular turgor pressure. Fungal spore cell walls consist almost exclusively of □-glucans (Noothalapati et al 2016, Scientific Reports 27789), and lack a distinct inner and outer wall structure.

The structure of plant pollen is distinct and varies from the structure of fungal spores in various aspects, for example, spores predominantly consist of □□ glucans wherein pollen comprise mostly cellulose, hemicellulose, callose and sporopollenin (Zimmermann et al. 2015, PLoS 10(4)).

The pore size of the spore cell wall has been studied for a few fungal species. Money and Webster (1988; Experimental Mycology 12 (2), 169-179) estimate the pore size in Achlya intricate to be approximately 2-3 nm.

It was therefore surprising that the beads for the magnetofection that have a diameter of 100 nm and with DNA coated upon them even 200 nm can deliver the DNA through the rigid cell wall of fungal spores resulting in a delivery of the DNA to the cells and allowing the generation of genetically manipulated fungal spores and cells.

The efficiency of deliver polynucleotides to the fungal spores is significantly increased when the polynucleotides, e.g. linear DNA or plasmid DNA, to be transfected are linked to a moiety susceptible to magnetic force of attraction, like MNPs, and said polynucleotide is delivered to the fungal spores to said cell by applying a magnetic field. The term “efficiency” for the method of the invention refers to the frequency of delivering polynucleotides, e.g. DNA, to a certain subset of the fungal spores. Increase of efficiency in delivery of the polynucleotides, e.g. linear or plasmid DNA, may be expressed in terms of increasing the frequency of transformation, or decreasing the time necessary for transferring a given amount of polynucleotides, e.g. DNA, into a given number of cells and/or in terms of increasing the amount of polynucleotides that is transferred into a given number of cells within a given time unit. Alternatively, transfection efficiency can be expressed in the form of the dose-response profile of a given polynucleotide. The term “dose-response profile” refers to the degree of an intended effect which is achievable per unit nucleic acid (or protein-DNA complex or nucleic acid analogon etc.) dose applied in a procedure in order to achieve such an effect. For gene transfer experiments, the term “dose-response profile” for example can relate to the level of expression of the transfected gene achievable per unit polynucleotide (e.g. DNA or RNA) dose applied in the transfection experiment or, for example, to the number of integration events in dependency of characteristics of the delivered polynucleotide.

It is understood herein, that with the term “introducing”, “delivering” or “transforming” polynucleotide(s) or nucleic acids into a fungal spore it is meant that RNA, DNA, e.g. linear DNA or plasmid DNA, double strand or single strand ribonucleotides, and nucleic acid analogon are introduced into the fungal spore. Consequently, the MNPs in the method of the present invention can be loaded with RNA, DNA, e.g. linear DNA or plasmid DNA, double strand or single strand ribonucleotides, nucleic acid analogon, or regulatory RNA, e.g. a microRNA, dsRNA, or antisence RNA. For example, the polynucleotide loaded to the MNPs is plasmid DNA or linear DNA and comprises a fungal regulatory element sequence, sequence encoding an polynucleotide that is active in a fungus, e.g. in the spore or in another life cycle stage, or a sequence derived from the fungus genome. The regulatory element is for example selected from the group consisting of a 5′-UTR, intron, terminator, enhancer, NEENA, and a promoter. The heterologous regulatory element can be e.g. from the same fungal species as the one that is transfected or from another species as long as it is functional during a phase or stage of the fungus' life cycle.

Thus, in one embodiment of the methods of the invention the polynucleotide molecules comprise DNA or RNA or nucleic acid analogons. The analogons, DNA or RNA molecules may be single stranded or double stranded, they may be linear or circular. For example, the polynucleotide molecules of the invention may be DNA encoding at least sequence, e.g. a regulatory element, a sequence included or derived from the fungal genome, a gene of interest functionally linked to a promoter, and/or a sequence being functional in the respective fungal spore or fungal cell derived therefrom. In another example, the polynucleotide molecule may be regulatory RNA, for example microRNA, antisense or dsRNA molecules for inducing RNAi in the respective fungal spore or fungal cell derived therefrom.

In one embodiment, the present invention refers to a method for the transformation a fungi comprising the steps of the method described therein, e.g. exposing a mixture of fungal spores and magnetic nanoparticles carrying the polypeptide to a magnet and/or a magnetic field, for example as described in steps (a) to (e), and allowing the integration of said polynucleotide molecules into the genome of said spores, thereby transforming the spore. The polynucleotide or a fragment thereof can be transiently be maintained in the cells or be stably integrated in to the genome of the spore DNA. The polynucleotide can be integrated into the genome of the fungus, e.g. or remains as plasmid in the cell or spore.

In one embodiment, in the method of invention, a fungus culture is grown from the fungal spore. Advantageously, the production of fungi from a transformed spore, e.g. a stable transformed spore, allows the generation of a new fungal strain with new traits and/or features. The method for the production of a transformed fungus can comprise the methods of the invention, e.g. comprising the steps of
a. loading polynucleotide molecules to be transferred into the spores on magnetic nanoparticles, and
b. adding said polynucleotide molecules loaded on said magnetic nanoparticles to said spores, and
c. exposing the mixture to a magnet, and
d. incubating the mixture in presence of said magnet to allow integration of said DNA molecules into the genome of said spores, and
e. growing fungal cells from said spores, thereby transforming the fungi.

After selection and/or after growing the fungal cells, cells that have said polynucleotide present are selected. In one embodiment, the polynucleotide or a fragment thereof is stably integrated into the genome of said spores. The polynucleotide can also be present as a plasmid. In one embodiment, the fungal spore and/or cell is thereby stably transformed. The polynucleotide or a fragment thereof can stably integrated in to the genome of the spore DNA. The fungal spores can provided before step a.

It was found that the spore density influences the efficiency of the transfection and transformation of the spore according to the method of the present invention. Thus, in one embodiment, the number of spores per millilitre in the incubation step is 10⁵ or higher, e.g. 10⁶ or higher, preferably, 10⁷ or higher.

Further, the efficiency of the method of the present invention can be improved by providing a preferred spore density in the incubation step per amount of polynucleotide. Thus, for example, the spore density is more than 10⁵/ml per 100-500 ng polynucleotide, e.g., it is more than 150 ng, 200 ng, or around 250 ng and less than 500 ng, less than 400 ng, e.g. around 300 ng. Thus, in one embodiment the amount of polynucleotide is between 200 ng and 300 ng of DNA, e.g. 250 ng DNA.

The incubation with the loaded MNPs, e.g. delivery of the DNA and/or the transfection reaction, is carried out for more than 0 min, 10 min, e.g. 20 min, or 30 min and less than 24 h, 20 h, 16 h, 12 h, 8 h, 6 h or 2 h. In addition, increases may also be obtained when applying the method in transfections for more than 30 min and less than 2 h.

In one embodiment, the incubation period starts with the appearance of the germ tube or 5 min, 10 min, 15 min, 20 min, 30 min, 25 min, 60 min, 75 min, 90 min, or 120 min or more and less than 48 h, less than 24 h, less than 12 h. less than 6 h, less than 2 h, less than 90 in, after the germ tube appeared. Thus, the germination time for the fungal spore before the transformation is for example between 0 h and 12 h, e.g. between 5 min and 6 h, e.g. between 10 min and 2 h after germination.

Thus, in one embodiment, the method of the invention uses a spore density of 10⁵ to 10⁷ spores per milliliter, 200 ng to 300 ng DNA in an incubation for 10 min to 30 min. For example, the method of the invention uses a spore density of around 10⁷ spores per milliliter, around 250 ng DNA in an incubation for 30 min.

In a further embodiment of the methods of the invention the DNA molecules introduced into the spores are integrated into the fungal genome, thereby generating stable transformed fungal cells or the DNA molecules are not integrated into the fungal genome, thereby generating transiently transformed fungal spores and/or fungal cells.

In another embodiment of the invention, the magnetic field in e.g. as in step d, of the methods of the invention is between 0.1 to 0.6T, between 0.2 and 0.5T, preferably the magnetic field is 0.3 T. The incubation time of the spores in the magnetic field in step e of the methods of the invention is between 5 and 60 min, between 10 and 50 min, between 20 and 40 min, preferably the incubation time is 30 min.

In one embodiment of the methods of the invention the magnetic nanoparticles comprise Fe3O4 or Fe2O3, preferably Fe3O4. In a further embodiment of the methods of the invention the magnetic nanoparticles comprise a magnetic core and a positively charged shell wherein the positively charged shell comprises or consists of polyethyleneimine (PEI). In a further embodiment the coated magnetic nanoparticles have a charge between +30 and +60 mV, between +35 and +55 mV or between +40 and +50 mV. Preferably the coated magnetic nanoparticles have a charge of +48.2 mV.

In one embodiment of the methods of the invention the average diameter of the coated magnetic nanoparticle is from 50 to 200 nm, from 70 to 180 nm or from 80 to 130 nm. Preferably the average diameter of the coated magnetic nanoparticle is from 100 to 120 nm.

In one embodiment, the present invention relates to a kit for transformation of fungal spores with polynucleotides comprising MNPs loaded with the polynucleotide in a magnetofection. The kit for example comprises one or more buffers or the means to produce buffers that allow the loading of the MNPs with den polynucleotides or the incubation of the fungal spores with the MNPs loaded with the polynucleotide. The kit for example comprises a device to apply the magnetic field to the spores. The device can for example be a devise that comprises one or more magnets closely located to containers, like wells in a plate or test tubes, that comprise the incubation buffer with loaded MNPs and fungal spores.

In one embodiment, the present invention also relates to a system for transformation of fungal spores with a polynucleotide comprising magnetic nanoparticles loaded with nucleic acid molecules. The system may comprise a devise to apply a magnetic field to the composition comprising the loaded spores and the MNPs. The system can also comprise a read-out devise that can measure the transfection efficiency achieved by the method of the invention, e.g. by measuring the activity or presence of a reporter gene or resistance to a selection.

Further, the present invention relates to a composition comprising MNPs loaded with nucleic acid molecules, and fungal spores. The number of spores, the density and the amount of polynucleotide, e.g. DNA, depends on the specific fungal species, DNA species and magnetic field. The composition of the invention allows for example a spore density in the composition of more than 10⁵/ml, 10⁶/ml or 10⁷/ml, e.g. between 10⁶/ml and 10⁷/ml, that is then used for an amount transfection with 100 ng to 500 ng polynucleotide, e.g. 200 ng to 300 ng polynucleotide, for example DNA.

In the kit, the composition or the system, the magnetic nanoparticles have the features as defined above. Further, the polynucleotide as used in the method of the invention, the kit of the invention, the system of the invention or the composition of the invention can be RNA, DNA, e.g. linear DNA or plasmid DNA, double strand or single strand ribonucleotides, and/or a nucleic acid analogon.

Further, in one embodiment, in the method or the system, or the kit or the composition of the invention, the polynucleotide loaded to the MNPs is Plasmid DNA or linear DNA, for example comprising a regulatory element, a sequence included or derived from the fungal genome, a gene of interest functionally linked to a promoter, and/or a sequence being functional in the respective fungal spore or fungal cell derived therefrom, or a RNA, e.g. a iRNA, microRNA, dsRNA, or antisense RNA.

In one embodiment, the fungal spores provided in the method the invention, the kit of the invention, the system of the invention or the composition of the invention are freshly harvested or already germinated, for example the germination time of the fungal spore before incubation with the loaded MNPs is between 0 h and 24 h, e.g. 0.5 h, 1 h, 2 h or 24 h.

The fungal spores to be transformed in the invention may be derived from any fungi. Preferably they are derived from Phakopsora spec, e.g. Phakopsora pachyrhizi, Zymoseptoria spec, e.g. Zymoseptoria tritici, Septoria, Mycosphaerella, Phythopthora spec., e.g. Phytopthora infestans, Puccinia, Sphaerotheca, Blumeria, Erysiphe, Alternaria, Botrytis, Ustilago, Venturia, Verticillium, Pyricularia, Magnaporthe, Plasmopara, Pythium, Sclerotinia, Colletotrichum, Penicillium, Neurospora, Aspergillus, Ashbya or Penicillium.

### Definitions

It is to be understood that this invention is not limited to the particular methodology or protocols. It is also to be understood that the terminology used herein is for the purpose of describing particular embodiments only, and is not intended to limit the scope of the present invention which will be limited only by the appended claims. It must be noted that as used herein and in the appended claims, the singular forms “a,” “and,” and “the” include plural reference unless the context clearly dictates otherwise. Thus, for example, reference to “a vector” is a reference to one or more vectors and includes equivalents thereof known to those skilled in the art, and so forth. The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent, preferably 10 percent up or down (higher or lower). As used herein, the word “or” means any one member of a particular list and also includes any combination of members of that list. The words “comprise,” “comprising,” “include,” “including,” and “includes” when used in this specification and in the following claims are intended to specify the presence of one or more stated features, integers, components, or steps, but they do not preclude the presence or addition of one or more other features, integers, components, steps, or groups thereof. For clarity, certain terms used in the specification are defined and used as follows:

Coding region: As used herein the term “coding region” when used in reference to a structural gene refers to the nucleotide sequences which encode the amino acids found in the nascent polypeptide as a result of translation of a mRNA molecule. The coding region is bounded, in eukaryotes, on the 5′-side by the nucleotide triplet “ATG” which encodes the initiator methionine, prokaryotes also use the triplets “GTG” and “TTG” as start codon. On the 3′-side it is bounded by one of the three triplets which specify stop codons (i.e., TAA, TAG, TGA). In addition, a gene may include sequences located on both the 5′- and 3′-end of the sequences which are present on the RNA transcript. These sequences are referred to as “flanking” sequences or regions (these flanking sequences are located 5′ or 3′ to the non-translated sequences present on the mRNA transcript). The 5′-flanking region may contain regulatory sequences such as promoters and enhancers which control or influence the transcription of the gene. The 3′-flanking region may contain sequences which direct the termination of transcription, post-transcriptional cleavage and polyadenylation.

Complementary: “Complementary” or “complementarity” refers to two nucleotide sequences which comprise antiparallel nucleotide sequences capable of pairing with one another (by the base-pairing rules) upon formation of hydrogen bonds between the complementary base residues in the antiparallel nucleotide sequences. For example, the sequence 5′-AGT-3′ is complementary to the sequence 5′-ACT-3′. Complementarity can be “partial” or “total.” “Partial” complementarity is where one or more nucleic acid bases are not matched according to the base pairing rules. “Total” or “complete” complementarity between nucleic acid molecules is where each and every nucleic acid base is matched with another base under the base pairing rules. The degree of complementarity between nucleic acid molecule strands has significant effects on the efficiency and strength of hybridization between nucleic acid molecule strands. A “complement” of a nucleic acid sequence as used herein refers to a nucleotide sequence whose nucleic acid molecules show total complementarity to the nucleic acid molecules of the nucleic acid sequence.

Endogenous: An “endogenous” nucleotide sequence refers to a nucleotide sequence, which is present in the genome of a wild type microorganism.

Enhanced expression: “enhance” or “increase” the expression of a nucleic acid molecule in a microorganism are used equivalently herein and mean that the level of expression of a nucleic acid molecule in a microorganism is higher compared to a reference microorganism, for example a wild type. The terms “enhanced” or “increased” as used herein mean herein higher, preferably significantly higher expression of the nucleic acid molecule to be expressed. As used herein, an “enhancement” or “increase” of the level of an agent such as a protein, mRNA or RNA means that the level is increased relative to a substantially identical microorganism grown under substantially identical conditions. As used herein, “enhancement” or “increase” of the level of an agent, such as for example a preRNA, mRNA, rRNA, tRNA, expressed by the target gene and/or of the protein product encoded by it, means that the level is increased 50% or more, for example 100% or more, preferably 200% or more, more preferably 5 fold or more, even more preferably 10 fold or more, most preferably 20 fold or more for example 50 fold relative to a suitable reference microorganism. The enhancement or increase can be determined by methods with which the skilled worker is familiar. Thus, the enhancement or increase of the nucleic acid or protein quantity can be determined for example by an immunological detection of the protein. Moreover, techniques such as protein assay, fluorescence, Northern hybridization, densitometric measurement of nucleic acid concentration in a gel, nuclease protection assay, reverse transcription (quantitative RT-PCR), ELISA (enzyme-linked immunosorbent assay), Western blotting, radioimmunoassay (RIA) or other immunoassays and fluorescence-activated cell analysis (FACS) can be employed to measure a specific protein or RNA in a microorganism. Depending on the type of the induced protein product, its activity or the effect on the phenotype of the microorganism may also be determined. Methods for determining the protein quantity are known to the skilled worker. Examples, which may be mentioned, are: the micro-Biuret method (Goa J (1953) Scand J Clin Lab Invest 5:218-222), the Folin-Ciocalteau method (Lowry O H et al. (1951) J Biol Chem 193:265-275) or measuring the absorption of CBB G-250 (Bradford M M (1976) Analyt Biochem 72:248-254).

Expression: “Expression” refers to the biosynthesis of a gene product, preferably to the transcription and/or translation of a nucleotide sequence, for example an endogenous gene or a heterologous gene, in a cell. For example, in the case of a structural gene, expression involves transcription of the structural gene into mRNA and—optionally—the subsequent translation of mRNA into one or more polypeptides. In other cases, expression may refer only to the transcription of the DNA harboring an RNA molecule.

Foreign: The term “foreign” refers to any nucleic acid molecule (e.g., gene sequence) which is introduced into a cell by experimental manipulations and may include sequences found in that cell as long as the introduced sequence contains some modification (e.g., a point mutation, the presence of a selectable marker gene, etc.) and is therefore different relative to the naturally-occurring sequence.

Functional fragment: the term “functional fragment” refers to any nucleic acid and/or protein which comprises merely a part of the full length nucleic acid and/or full length polypeptide of the invention but still provides the same function, i.e. the function of an AAT enzyme catalyzing the reaction of acryloyl-CoA and butanol to n-BA and CoA. Preferably, the fragment comprises at least 50%, at least 60%, at least 70%, at least 80%, at least 90% at least 95%, at least 98%, at least 99% of the sequence from which it is derived. Preferably, the functional fragment comprises contiguous nucleic acids or amino acids of the nucleic acid and/or protein from which the functional fragment is derived. A functional fragment of a nucleic acid molecule encoding a protein means a fragment of the nucleic acid molecule encoding a functional fragment of the protein.

Functional linkage: The term “functional linkage” or “functionally linked” is equivalent to the term “operable linkage” or “operably linked” and is to be understood as meaning, for example, the sequential arrangement of a regulatory element (e.g. a promoter) with a nucleic acid sequence to be expressed and, if appropriate, further regulatory elements (such as e.g., a terminator) in such a way that each of the regulatory elements can fulfill its intended function to allow, modify, facilitate or otherwise influence expression of said nucleic acid sequence. As a synonym the wording “operable linkage” or “operably linked” may be used. The expression may result depending on the arrangement of the nucleic acid sequences in relation to sense or antisense RNA. To this end, direct linkage in the chemical sense is not necessarily required. Genetic control sequences such as, for example, enhancer sequences, can also exert their function on the target sequence from positions which are further away, or indeed from other DNA molecules. Preferred arrangements are those in which the nucleic acid sequence to be expressed recombinantly is positioned behind the sequence acting as promoter, so that the two sequences are linked covalently to each other. In a preferred embodiment, the nucleic acid sequence to be transcribed is located behind the promoter in such a way that the transcription start is identical with the desired beginning of the chimeric RNA of the invention.

Functional linkage, and an expression construct, can be generated by means of customary recombination and cloning techniques as described (e.g., Sambrook J, Fritsch E F and Maniatis T (1989); Silhavy et al. (1984) Experiments with Gene Fusions, Cold Spring Harbor Laboratory, Cold Spring Harbor (N.Y.); Ausubel et al. (1987) Current Protocols in Molecular Biology, Greene Publishing Assoc. and Wiley Interscience, Gelvin et al. (Eds) (1990) Plant Molecular Biology Manual; Kluwer Academic Publisher, Dordrecht, The Netherlands). However, further sequences, which, for example, act as a linker with specific cleavage sites for restriction enzymes, or as a signal peptide, may also be positioned between the two sequences. The insertion of sequences may also lead to the expression of fusion proteins. Preferably, the expression construct, consisting of a linkage of a regulatory region for example a promoter and nucleic acid sequence to be expressed, can exist in a vector-integrated form or can be inserted into the genome, for example by transformation.

Gene: The term “gene” refers to a region operably linked to appropriate regulatory sequences capable of regulating the expression of the gene product (e.g., a polypeptide or a functional RNA) in some manner. A gene includes untranslated regulatory regions of DNA (e.g., promoters, enhancers, repressors, etc.) preceding (up-stream) and following (downstream) the coding region (open reading frame, ORF). The term “structural gene” as used herein is intended to mean a DNA sequence that is transcribed into mRNA which is then translated into a sequence of amino acids characteristic of a specific polypeptide.

Genome and genomic DNA: The terms “genome” or “genomic DNA” is referring to the heritable genetic information of a host organism. Said genomic DNA comprises the DNA of the nucleoid but also the DNA of the self-replicating plasmid.

Heterologous: The term “heterologous” with respect to a nucleic acid molecule or DNA refers to a nucleic acid molecule which is operably linked to, or is manipulated to become operably linked to, a second nucleic acid molecule to which it is not operably linked in nature, or to which it is operably linked at a different location in nature. A heterologous expression construct comprising a nucleic acid molecule and one or more regulatory nucleic acid molecule (such as a promoter or a transcription termination signal) linked thereto for example is a constructs originating by experimental manipulations in which either a) said nucleic acid molecule, or b) said regulatory nucleic acid molecule or c) both (i.e. (a) and (b)) is not located in its natural (native) genetic environment or has been modified by experimental manipulations, an example of a modification being a substitution, addition, deletion, inversion or insertion of one or more nucleotide residues. Natural genetic environment refers to the natural genomic locus in the organism of origin, or to the presence in a genomic library. In the case of a genomic library, the natural genetic environment of the sequence of the nucleic acid molecule is preferably retained, at least in part. The environment flanks the nucleic acid sequence at least at one side and has a sequence of at least 50 bp, preferably at least 500 bp, especially preferably at least 1,000 bp, very especially preferably at least 5,000 bp, in length. A naturally occurring expression construct—for example the naturally occurring combination of a promoter with the corresponding gene—becomes a transgenic expression construct when it is modified by non-natural, synthetic “artificial” methods such as, for example, mutagenization. Such methods have been described (U.S. Pat. No. 5,565,350; WO 00/15815). For example, a protein encoding nucleic acid molecule operably linked to a promoter, which is not the native promoter of this molecule, is considered to be heterologous with respect to the promoter. Preferably, heterologous DNA is not endogenous to or not naturally associated with the cell into which it is introduced but has been obtained from another cell or has been synthesized. Heterologous DNA also includes an endogenous DNA sequence, which contains some modification, non-naturally occurring, multiple copies of an endogenous DNA sequence, or a DNA sequence which is not naturally associated with another DNA sequence physically linked thereto. Generally, although not necessarily, heterologous DNA encodes RNA or proteins that are not normally produced by the cell into which it is expressed.

Hybridization: The term “hybridization” as used herein includes “any process by which a strand of nucleic acid molecule joins with a complementary strand through base pairing.” (J. Coombs (1994) Dictionary of Biotechnology, Stockton Press, New York). Hybridization and the strength of hybridization (i.e., the strength of the association between the nucleic acid molecules) is impacted by such factors as the degree of complementarity between the nucleic acid molecules, stringency of the conditions involved, the Tm of the formed hybrid, and the G:C ratio within the nucleic acid molecules. As used herein, the term “Tm” is used in reference to the “melting temperature.” The melting temperature is the temperature at which a population of double-stranded nucleic acid molecules becomes half dissociated into single strands. The equation for calculating the Tm of nucleic acid molecules is well known in the art. As indicated by standard references, a simple estimate of the Tm value may be calculated by the equation: Tm=81.5+0.41(% G+C), when a nucleic acid molecule is in aqueous solution at 1 M NaCl [see e.g., Anderson and Young, Quantitative Filter Hybridization, in Nucleic Acid Hybridization (1985)]. Other references include more sophisticated computations, which take structural as well as sequence characteristics into account for the calculation of Tm. Stringent conditions are known to those skilled in the art and can be found in Current Protocols in Molecular Biology, John Wiley & Sons, N.Y. (1989), 6.3.1-6.3.6.

Suitable hybridization conditions are for example hybridizing under conditions equivalent to hybridization in 7% sodium dodecyl sulfate (SDS), 0.5 M NaPO4, 1 mM EDTA at 50° C. with washing in 2×SSC, 0.1% SDS at 50° C. (low stringency) to a nucleic acid molecule comprising at least 50, preferably at least 100, more preferably at least 150, even more preferably at least 200, most preferably at least 250 consecutive nucleotides of the complement of a sequence. Other suitable hybridizing conditions are hybridization in 7% sodium dodecyl sulfate (SDS), 0.5 M NaPO4, 1 mM EDTA at 50° C. with washing in 1×SSC, 0.1% SDS at 50° C. (medium stringency) or 65° C. (high stringency) to a nucleic acid molecule comprising at least 50, preferably at least 100, more preferably at least 150, even more preferably at least 200, most preferably at least 250 consecutive nucleotides of a complement of a sequence. Other suitable hybridization conditions are hybridization in 7% sodium dodecyl sulfate (SDS), 0.5 M NaPO4, 1 mM EDTA at 50° C. with washing in 0.1×SSC, 0.1% SDS at 65° C. (very high stringency) to a nucleic acid molecule comprising at least 50, preferably at least 100, more preferably at least 150, even more preferably at least 200, most preferably at least 250 consecutive nucleotides of a complement of a sequence.

“Identity”: “Identity” when used in respect to the comparison of two or more nucleic acid or amino acid molecules means that the sequences of said molecules share a certain degree of sequence similarity, the sequences being partially identical.

For the determination of the percentage identity of two or more amino acids or of two or more nucleotide sequences several computer software programs have been developed. The identity of two or more sequences can be calculated with for example the software fasta, which presently has been used in the version fasta 3 (W. R. Pearson and D. J. Lipman, PNAS 85, 2444(1988); W. R. Pearson, Methods in Enzymology 183, 63 (1990); W. R. Pearson and D. J. Lipman, PNAS 85, 2444 (1988); W. R. Pearson, Enzymology 183, 63 (1990)). Another useful program for the calculation of identities of different sequences is the standard blast program, which is included in the Biomax pedant software (Biomax, Munich, Federal Republic of Germany). This leads unfortunately sometimes to suboptimal results since blast does not always include complete sequences of the subject and the query. Nevertheless, as this program is very efficient it can be used for the comparison of a huge number of sequences. The following settings are typically used for such a comparison of sequences:

-p Program Name [String]; -d Database [String]; default=nr; -i Query File [File In]; default=stdin; -e Expectation value (E) [Real]; default=10.0; -m alignment view options: 0=pairwise; 1=query-anchored showing identities; 2=query-anchored no identities; 3=flat query-anchored, show identities; 4=flat query-anchored, no identities; 5=query-anchored no identities and blunt ends; 6=flat query-anchored, no identities and blunt ends; 7=XML Blast output; 8=tabular; 9 tabular with comment lines [Integer]; default=0; -o BLAST report Output File [File Out] Optional; default=stdout; -F Filter query sequence (DUST with blastn, SEG with others) [String]; default=T; -G Cost to open a gap (zero invokes default behavior) [Integer]; default=0; -E Cost to extend a gap (zero invokes default behavior) [Integer]; default=0; -X X dropoff value for gapped alignment (in bits) (zero invokes default behavior); blastn 30, megablast 20, tblastx 0, all others 15 [Integer]; default=0; -I Show GI's in deflines [T/F]; default=F; -q Penalty for a nucleotide mismatch (blastn only) [Integer]; default=−3; -r Reward for a nucleotide match (blastn only) [Integer]; default=1; -v Number of database sequences to show one-line descriptions for (V) [Integer]; default=500; -b Number of database sequence to show alignments for (B) [Integer]; default=250; -f Threshold for extending hits, default if zero; blastp 11, blastn 0, blastx 12, tblastn 13; tblastx 13, megablast 0 [Integer]; default=0; -g Perfom gapped alignment (not available with tblastx) [T/F]; default=T; -Q Query Genetic code to use [Integer]; default=1; -D DB Genetic code (for tblast[nx] only) [Integer]; default=1; -a Number of processors to use [Integer]; default=1; -O SeqAlign file [File Out] Optional; -J Believe the query defline [T/F]; default=F; -M Matrix [String]; default=BLOSUM62; -W Word size, default if zero (blastn 11, megablast 28, all others 3) [Integer];

default=0; -z Effective length of the database (use zero for the real size) [Real]; default=0; -K Number of best hits from a region to keep (off by default, if used a value of 100 is recommended) [Integer]; default=0; -P 0 for multiple hit, 1 for single hit [Integer]; default=0; -Y Effective length of the search space (use zero for the real size) [Real]; default=0; -S Query strands to search against database (for blast[nx], and tblastx); 3 is both, 1 is top, 2 is bottom [Integer]; default=3; -T Produce HTML output [T/F]; default=F; -I Restrict search of database to list of GI's [String] Optional; -U Use lower case filtering of FASTA sequence [T/F] Optional; default=F; -y X dropoff value for ungapped extensions in bits (0.0 invokes default behavior); blastn 20, megablast 10, all others 7 [Real]; default=0.0; -Z X dropoff value for final gapped alignment in bits (0.0 invokes default behavior); blastn/megablast 50, tblastx 0, all others 25 [Integer]; default=0; -R PSI-TBLASTN checkpoint file [File In] Optional; -n MegaBlast search [T/F]; default=F; -L Location on query sequence [String] Optional; -A Multiple Hits window size, default if zero (blastn/megablast 0, all others 40 [Integer]; default=0; -w Frame shift penalty (OOF algorithm for blastx) [Integer]; default=0; -t Length of the largest intron allowed in tblastn for linking HSPs (0 disables linking) [Integer]; default=0.

Results of high quality are reached by using the algorithm of Needleman and Wunsch or Smith and Waterman. Therefore, programs based on said algorithms are preferred. Advantageously the comparisons of sequences can be done with the program PileUp (J. Mol. Evolution, 25, 351 (1987), Higgins et al., CABIOS 5, 151 (1989)) or preferably with the programs “Gap” and “Needle”, which are both based on the algorithms of Needleman and Wunsch (J. Mol. Biol. 48; 443 (1970)), and “BestFit”, which is based on the algorithm of Smith and Waterman (Adv. Appl. Math. 2; 482 (1981)). “Gap” and “BestFit” are part of the GCG software-package (Genetics Computer Group, 575 Science Drive, Madison, Wis., USA 53711 (1991); Altschul et al., (Nucleic Acids Res. 25, 3389 (1997)), “Needle” is part of the The European Molecular Biology Open Software Suite (EMBOSS) (Trends in Genetics 16 (6), 276 (2000)). Therefore preferably the calculations to determine the percentages of sequence identity are done with the programs “Gap” or “Needle” over the whole range of the sequences. The following standard adjustments for the comparison of nucleic acid sequences were used for “Needle”: matrix: EDNAFULL, Gap_penalty: 10.0, Extend_penalty: 0.5. The following standard adjustments for the comparison of nucleic acid sequences were used for “Gap”: gap weight: 50, length weight: 3, average match: 10.000, average mismatch: 0.000.

For example, a sequence, which is said to have 80% identity with sequence SEQ ID NO: 1 at the nucleic acid level is understood as meaning a sequence which, upon comparison with the sequence represented by SEQ ID NO: 1 by the above program “Needle” with the above parameter set, has a 80% identity. Preferably the identity is calculated on the complete length of the query sequence, for example SEQ ID NO: 1.

Isolated: The term “isolated” as used herein means that a material has been removed by the hand of man and exists apart from its original, native environment and is therefore not a product of nature. An isolated material or molecule (such as a DNA molecule or enzyme) may exist in a purified form or may exist in a non-native environment such as, for example, in a transgenic host cell. For example, a naturally occurring nucleic acid molecule or polypeptide present in a living cell is not isolated, but the same nucleic acid molecule or polypeptide, separated from some or all of the coexisting materials in the natural system, is isolated. Such nucleic acid molecules can be part of a vector and/or such nucleic acid molecules or polypeptides could be part of a composition and would be isolated in that such a vector or composition is not part of its original environment. Preferably, the term “isolated” when used in relation to a nucleic acid molecule, as in “an isolated nucleic acid sequence” refers to a nucleic acid sequence that is identified and separated from at least one contaminant nucleic acid molecule with which it is ordinarily associated in its natural source. Isolated nucleic acid molecule is nucleic acid molecule present in a form or setting that is different from that in which it is found in nature. In contrast, non-isolated nucleic acid molecules are nucleic acid molecules such as DNA and RNA, which are found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs, which encode a multitude of proteins. However, an isolated nucleic acid sequence comprising for example SEQ ID NO: 1 includes, by way of example, such nucleic acid sequences in cells which ordinarily contain SEQ ID NO: 1 where the nucleic acid sequence is in a genomic or plasmid location different from that of natural cells, or is otherwise flanked by a different nucleic acid sequence than that found in nature. The isolated nucleic acid sequence may be present in single- or double-stranded form. When an isolated nucleic acid sequence is to be utilized to express a protein, the nucleic acid sequence will contain at a minimum at least a portion of the sense or coding strand (i.e., the nucleic acid sequence may be single-stranded). Alternatively, it may contain both the sense and anti-sense strands (i.e., the nucleic acid sequence may be double-stranded).

Non-coding: The term “non-coding” refers to sequences of nucleic acid molecules that do not encode part or all of an expressed protein. Non-coding sequences include but are not limited enhancers, promoter regions, 3′ untranslated regions, and 5′ untranslated regions.

Nucleic acids and nucleotides: The terms “nucleic acids” and “Nucleotides” refer to naturally occurring or synthetic or artificial nucleic acid or nucleotides. The terms “nucleic acids” and “nucleotides” comprise deoxyribonucleotides or ribonucleotides or any nucleotide analogue and polymers or hybrids thereof in either single- or double-stranded, sense or antisense form. Unless otherwise indicated, a particular nucleic acid sequence also implicitly encompasses conservatively modified variants thereof (e.g., degenerate codon substitutions) and complementary sequences, as well as the sequence explicitly indicated. The term “nucleic acid” is used inter-changeably herein with “gene”, “cDNA, “mRNA”, “oligonucleotide,” and “nucleic acid molecule”. Nucleotide analogues include nucleotides having modifications in the chemical structure of the base, sugar and/or phosphate, including, but not limited to, 5-position pyrimidine modifications, 8-position purine modifications, modifications at cytosine exocyclic amines, substitution of 5-bromo-uracil, and the like; and 2′-position sugar modifications, including but not limited to, sugar-modified ribonucleotides in which the 2′-OH is replaced by a group selected from H, OR, R, halo, SH, SR, NH2, NHR, NR2, or CN. Short hairpin RNAs (shRNAs) also can comprise non-natural elements such as non-natural bases, e.g., ionosin and xanthine, non-natural sugars, e.g., 2′-methoxy ribose, or non-natural phosphodiester linkages, e.g., methylphosphonates, phosphorothioates and peptides.

Nucleic acid sequence: The phrase “nucleic acid sequence” refers to a single- or double-stranded polymer of deoxyribonucleotide or ribonucleotide bases read from the 5′- to the 3′-end. It includes chromosomal DNA, self-replicating plasmids, infectious polymers of DNA or RNA and DNA or RNA that performs a primarily structural role. “Nucleic acid sequence” also refers to a consecutive list of abbreviations, letters, characters or words, which represent nucleotides. In one embodiment, a nucleic acid can be a “probe” which is a relatively short nucleic acid, usually less than 100 nucleotides in length. Often a nucleic acid probe is from about 50 nucleotides in length to about 10 nucleotides in length. A “target region” of a nucleic acid is a portion of a nucleic acid that is identified to be of interest. A “coding region” of a nucleic acid is the portion of the nucleic acid, which is transcribed and translated in a sequence-specific manner to produce into a particular polypeptide or protein when placed under the control of appropriate regulatory sequences. The coding region is said to encode such a polypeptide or protein.

Oligonucleotide: The term “oligonucleotide” refers to an oligomer or polymer of ribonucleic acid (RNA) or deoxyribonucleic acid (DNA) or mimetics thereof, as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases. An oligonucleotide preferably includes two or more nucleomonomers covalently coupled to each other by linkages (e.g., phosphodiesters) or substitute linkages.

Overhang: An “overhang” is a relatively short single-stranded nucleotide sequence on the 5′- or 3′-hydroxyl end of a double-stranded oligonucleotide molecule (also referred to as an “extension,” “protruding end,” or “sticky end”).

Polypeptide: The terms “polypeptide”, “peptide”, “oligopeptide”, “polypeptide”, “gene product”, “expression product” and “protein” are used interchangeably herein to refer to a polymer or oligomer of consecutive amino acid residues.

Promoter: The terms “promoter”, or “promoter sequence” are equivalents and as used herein, refer to a DNA sequence which when operably linked to a nucleotide sequence of interest is capable of controlling the transcription of the nucleotide sequence of interest into RNA. A promoter is located 5′ (i.e., upstream), proximal to the transcriptional start site of a nucleotide sequence of interest whose transcription into mRNA it controls and provides a site for specific binding by RNA polymerase and other transcription factors for initiation of transcription. The promoter does not comprise coding regions or 5′ untranslated regions. The promoter may for example be heterologous or homologous to the respective cell. A nucleic acid molecule sequence is “heterologous to” an organism or a second nucleic acid molecule sequence if it originates from a foreign species, or, if from the same species, is modified from its original form. For example, a promoter operably linked to a heterologous coding sequence refers to a coding sequence from a species different from that from which the promoter was derived, or, if from the same species, a coding sequence which is not naturally associated with the promoter (e.g. a genetically engineered coding sequence or an allele from a different ecotype or variety). Suitable promoters can be derived from genes of the host cells where expression should occur or from pathogens for this host.

Purified: As used herein, the term “purified” refers to molecules, either nucleic or amino acid sequences that are removed from their natural environment, isolated or separated. “Substantially purified” molecules are at least 60% free, preferably at least 75% free, and more preferably at least 90% free from other components with which they are naturally associated. A purified nucleic acid sequence may be an isolated nucleic acid sequence.

Significant increase: An increase for example in enzymatic activity, gene expression, productivity or yield of a certain product, that is larger than the margin of error inherent in the measurement technique, preferably an increase by about 10% or 25% preferably by 50% or 75%, more preferably 2-fold or-5 fold or greater of the activity, expression, productivity or yield of the control enzyme or expression in the control cell, productivity or yield of the control cell, even more preferably an increase by about 10-fold or greater.

Significant decrease: A decrease for example in enzymatic activity, gene expression, productivity or yield of a certain product, that is larger than the margin of error inherent in the measurement technique, preferably a decrease by at least about 5% or 10%, preferably by at least about 20% or 25%, more preferably by at least about 50% or 75%, even more preferably by at least about 80% or 85%, most preferably by at least about 90%, 95%, 97%, 98% or 99%. Substantially complementary: In its broadest sense, the term “substantially complementary”, when used herein with respect to a nucleotide sequence in relation to a reference or target nucleotide sequence, means a nucleotide sequence having a percentage of identity between the substantially complementary nucleotide sequence and the exact complementary sequence of said reference or target nucleotide sequence of at least 60%, more desirably at least 70%, more desirably at least 80% or 85%, preferably at least 90%, more preferably at least 93%, still more preferably at least 95% or 96%, yet still more preferably at least 97% or 98%, yet still more preferably at least 99% or most preferably 100% (the latter being equivalent to the term “identical” in this context). Preferably identity is assessed over a length of at least 19 nucleotides, preferably at least 50 nucleotides, more preferably the entire length of the nucleic acid sequence to said reference sequence (if not specified otherwise below). Sequence comparisons are carried out using default GAP analysis with the University of Wisconsin GCG, SEQWEB application of GAP, based on the algorithm of Needleman and Wunsch (Needleman and Wunsch (1970) J Mol. Biol. 48: 443-453; as defined above). A nucleotide sequence “substantially complementary” to a reference nucleotide sequence hybridizes to the reference nucleotide sequence under low stringency conditions, preferably medium stringency conditions, most preferably high stringency conditions (as defined above).

Transgene: The term “transgene” as used herein refers to any nucleic acid sequence, which is introduced into the genome of a cell by experimental manipulations. A transgene may be an “endogenous DNA sequence,” or a “heterologous DNA sequence” (i.e., “foreign DNA”). The term “endogenous DNA sequence” refers to a nucleotide sequence, which is naturally found in the cell into which it is introduced so long as it does not contain some modification (e.g., a point mutation, the presence of a selectable marker gene, etc.) relative to the naturally-occurring sequence.

Transgenic: The term transgenic when referring to an organism means transformed, preferably stably transformed, with at least one recombinant nucleic acid molecule.

Vector: As used herein, the term “vector” refers to a nucleic acid molecule capable of transporting another nucleic acid molecule to which it has been linked. One type of vector is a genomic integrated vector, or “integrated vector”, which can become integrated into the genomic DNA of the host cell. Another type of vector is an episomal vector, i.e., a plasmid or a nucleic acid molecule capable of extra-chromosomal replication. Vectors capable of directing the expression of genes to which they are operatively linked are referred to herein as “expression vectors”. In the present specification, “plasmid” and “vector” are used interchangeably unless otherwise clear from the context.

Wild type: The term “wild type”, “natural” or “natural origin” means with respect to an organism that said organism is not changed, mutated, or otherwise manipulated by man. With respect to a polypeptide or nucleic acid sequence, that the polypeptide or nucleic acid sequence is naturally occurring or available in at least one naturally occurring organism which is not changed, mutated, or otherwise manipulated by man.

A wild type of a microorganism refers to a microorganism whose genome is present in a state as before the introduction of a genetic modification of a certain gene. The genetic modification may be e.g. a deletion of a gene or a part thereof or a point mutation or the introduction of a gene.

The terms “production” or “productivity” are art-recognized and include the concentration of the fermentation product (for example, dsRNA) formed within a given time and a given fermentation volume (e.g., kg product per hour per liter). The term “efficiency of production” includes the time required for a particular level of production to be achieved (for example, how long it takes for the cell to attain a particular rate of output of a fine chemical).

The term “yield” or “product/carbon yield” is art-recognized and includes the efficiency of the conversion of the carbon source into the product (i.e., fine chemical). This is generally written as, for example, kg product per kg carbon source. By increasing the yield or production of the compound, the quantity of recovered molecules or of useful recovered molecules of that compound in a given amount of culture over a given amount of time is increased.

The term “recombinant microorganism” includes microorganisms which have been genetically modified such that they exhibit an altered or different genotype and/or phenotype (e. g., when the genetic modification affects coding nucleic acid sequences of the microorganism) as compared to the wild type microorganism from which it was derived. A recombinant microorganism comprises at least one recombinant nucleic acid molecule.

The term “recombinant” with respect to nucleic acid molecules refers to nucleic acid molecules produced by man using recombinant nucleic acid techniques. The term comprises nucleic acid molecules which as such do not exist in nature or do not exist in the organism from which the nucleic acid molecule is derived, but are modified, changed, mutated or otherwise manipulated by man. Preferably, a “recombinant nucleic acid molecule” is a non-naturally occurring nucleic acid molecule that differs in sequence from a naturally occurring nucleic acid molecule by at least one nucleic acid. A “recombinant nucleic acid molecules” may also comprise a “recombinant construct” which comprises, preferably operably linked, a sequence of nucleic acid molecules not naturally occurring in that order. Preferred methods for producing said recombinant nucleic acid molecules may comprise cloning techniques, directed or non-directed mutagenesis, gene synthesis or recombination techniques.

An example of such a recombinant nucleic acid molecule is a plasmid into which a heterologous DNA-sequence has been inserted or a gene or promoter which has been mutated compared to the gene or promoter from which the recombinant nucleic acid molecule derived. The mutation may be introduced by means of directed mutagenesis technologies known in the art or by random mutagenesis technologies such as chemical, UV light or x-ray mutagenesis or directed evolution technologies.

The term “directed evolution” is used synonymously with the term “metabolic evolution” herein and involves applying a selection pressure that favors the growth of mutants with the traits of interest. The selection pressure can be based on different culture conditions, ATP and growth coupled selection and redox related selection. The selection pressure can be carried out with batch fermentation with serial transferring inoculation or continuous culture with the same pressure.

The term “expression” or “gene expression” means the transcription of a specific gene(s) or specific genetic vector construct. The term “expression” or “gene expression” in particular means the transcription of gene(s) or genetic vector construct into mRNA. The process includes transcription of DNA and may include processing of the resulting RNA-product. The term “expression” or “gene expression” may also include the translation of the mRNA and therewith the synthesis of the encoded protein, i.e. protein expression.

### FIGURES

FIG. 1: Vector pSJ+GFP(HPT)-MF

FIG. 2: Vector pSJ(basic)-MF

FIG. 3: Table 1: Overview results of magnetofection of fungal spores

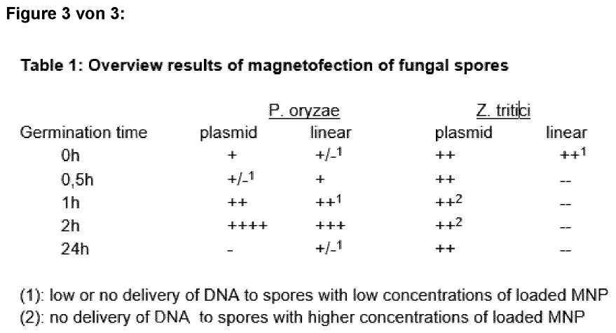

### EXAMPLES

### Chemicals and Common Methods

Unless indicated otherwise, cloning procedures carried out for the purposes of the present invention including restriction digest, agarose gel electrophoresis, purification of nucleic acids, ligation of nucleic acids, transformation, selection and cultivation of bacterial cells are performed as described (Sambrook J, Fritsch E F and Maniatis T (1989)). Sequence analyses of recombinant DNA are performed with a laser fluorescence DNA sequencer (Applied Biosystems, Foster City, Calif., USA) using the Sanger technology (Sanger et al., 1977). Unless described otherwise, chemicals and reagents are obtained from Sigma Aldrich (Sigma Aldrich, St. Louis, USA), from Promega (Madison, Wis., USA), Duchefa (Haarlem, The Netherlands) or Invitrogen (Carlsbad, Calif., USA). Restriction endonucleases are from New England Biolabs (Ipswich, Mass., USA) or Roche Diagnostics GmbH (Penzberg, Germany). Oligonucleotides are synthesized by Eurofins MWG Operon (Ebersberg, Germany).

### Example 1. Production of Nano Fe3O4/PEI Particles

Nano Fe3O4/PEI particles are made as described in patent application CN103233042.

### Example 2. Nano Fe3O4/PEI Gene Vector-Mediated Gene Transfer to Phakopsora pachyrhizi Spores

A vector, containing a fungal Uf-PMA1 promoter and -terminator (Djulic et al. Fungal Biology 115, 633-642 (2011)) driving both the SucDH1 (H254Y) gene associated with fungicide resistance and the DsRed reporter gene can be linearized by restriction enzyme digestion and mixed with magnetic nanoparticles, coated with PEI. Fungal spores from Phakospora pachyrhizi can be collected by gently tapping infected leaves and collecting spores.

Magnetic nanoparticles coated with PEI, can be mixed with plasmid DNA in a 1:2 ratio, i.e. 1 mg magnetic nanoparticles and 2 mg plasmid DNA, and subsequently added to a in 1 mL aqueous solution containing approximately 10⁶ spores. Then, this mixture can be put into a 0.3 T magnetic field with regular mixing.

Applying selection of resistant fungi following the procedure outlined in Djulic et al, ibid. using in planta selection with 50 mg/mL Carboxin treatment shows successful transformation of fungal spores.

### Example 3: Binding of DNA to MNPs

MNP and plasmid DNA were mixed to form complex (MNP/DNA complex) by the attraction between positive (MNP) and negative (DNA) charges. If MNPs are fully loaded by DNA, the MNP/DNA complex has no charge, and it will stay in the wells of a gel at an electrophoresis experiment. When there is more DNA than can be bound by the MNPs, it will run into the gel and DNA bands can be detected. The highest MNP/DNA ratio without DNA band detected determined the optimal ratio for MNP and plasmid DNA. The manufacturer of the Magnetofection technology offers two types of MNPs (PolyMAG and CombiMAG) and both were tested.

It was shown that the plasmid DNA did not bind to the CombiMAG beads and binds to the PolyMAG beads. Also, linear DNA derived from a PCR binds to PolyMAG beats.

### Example 4: Transformation of Pyricularia oryzae and Zymoseptoria tritici

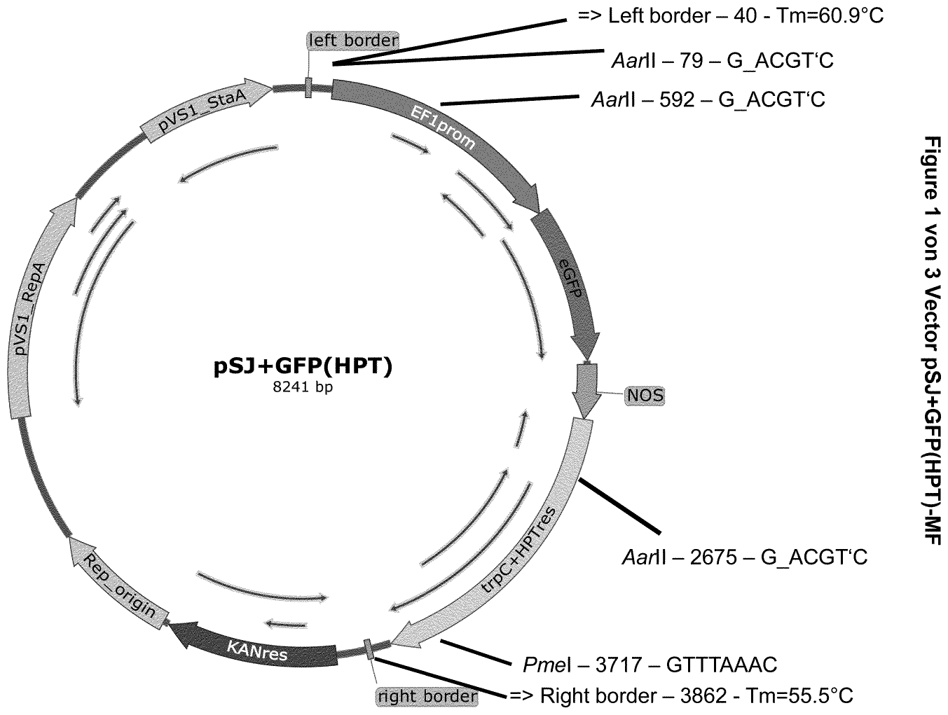

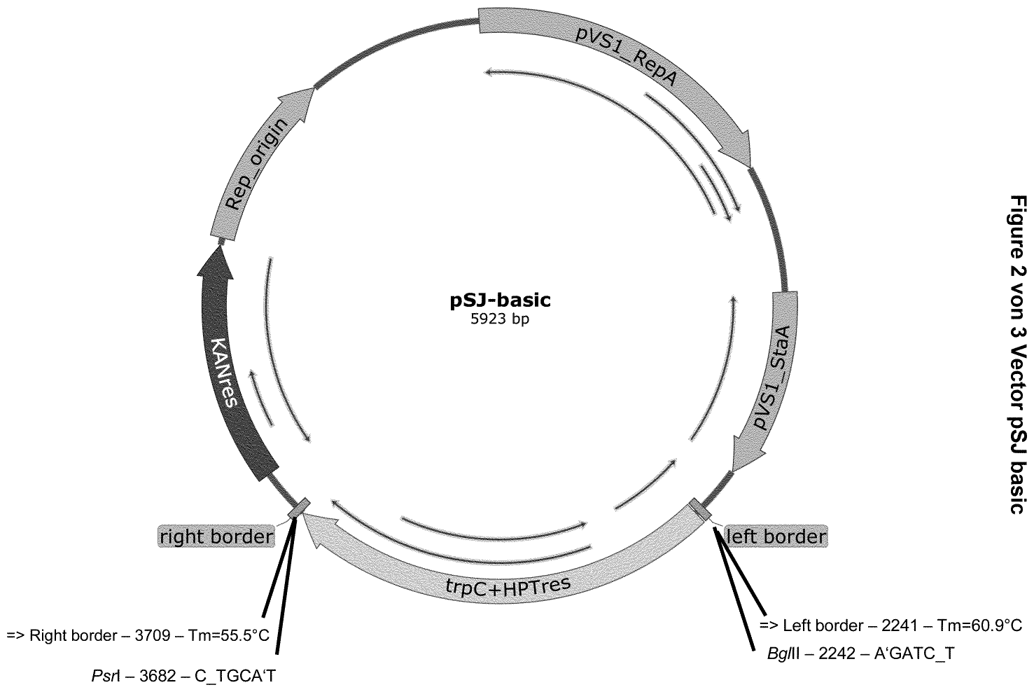

Two vectors with hygromycin as selection marker and carrying a GFP expression cassette were used. These vectors work well with the two fungi. The plasmids pSJ+GFP(HPT)-MF and pSJ(basic)-MF are shown in FIG. 1 and FIG. 2, respectively. Binding-time of magnetic beads to DNA 0.5 h, 1 h and 2 h at 4° C. were tested in a 96-well-plate: 200 μl/well (volume of DNA+MNPs) and in a 24-well-plate: 500 μl/well (volume of DNA+MNPs). The DNA amount/well was set up to 250 ng/well. Fungal spore densities of Magnaporthe and Zymoseptoria of 10⁴/ml, 10⁵/ml, 10⁶/ml, and 10⁷/ml were tested.

Transfection were successful with binding times for the loading of the DNA to the MNPs of 1 h or 2 h, spore densities of 10⁵/ml, 10⁶/ml, or 10⁷/ml and incubation times with spores of 30 min on the magnetic plate.

### Example 5: Results of Delivery of DNA to Spores from Zymoseptoria tritici

Successful delivery of DNA to spores were achieved with plasmid DNA with fungal spores germinated for 0 h, 0.5 h, 1 h, 2 h and 24 h. Linear DNA can be delivered to spores freshly harvested if the DNA/MNP complex concentration is increased for incubation with the spores. Delivery of DNA to the spores is found for spore densities of 10⁵/ml, 10⁶/ml, and 10⁷/ml, with the transformation rates increasing with the increase of the density of spores. For example, the transformation rates were at 10⁵/ml lower than 10⁶/ml, and lower than 10⁷/ml.

### Example 6: Germination Times and Transformation Efficiency

Delivery of DNA to the spores were observed if magnetofection occurred 0 h, 0.5 h, 1 h, 2 h, or 24 h after start of germination of the fungal spores for spore densities of 10⁵/ml, 10⁶/ml, and 10⁷/ml. The highest delivery of DNA to the spores were observed at higher densities and at 0 h germination time.

In southern blots the integration of the DNA into the genome could be confirmed, with a transformation rate that is lower than in Agrobacterium mediated transformation).

### Example 7: Results of Delivery of DNA to Spores from Pyricularia oryzae

Successful delivery of DNA to spores were achieved with plasmid DNA with fungal spores germinated for 0 h, 0.5 h, 1 h, 2 h. Linear DNA can be delivered to spores germinated for 0 h, 0.5 h, 1 h, 2 h and 24 h if the DNA/MNP complex concentration is increased for incubation with the spores. Highest delivery rates for linear DNA as well as plasmid DNA were achieved after 2 h of germination, independent from DNA/MNP complex density. Delivery of DNA to the spores is found for spore densities of 10⁵/ml, 10⁶/ml, and 10⁷/ml, with the transformation rates increasing with the increase of the density of spores. For example, the transformation rates were at 10⁵/ml lower than 10⁶/ml, and lower than 10⁷/ml.

1. A method for introducing polynucleotide molecules into fungal spores comprising exposing a mixture of fungal spores and magnetic nanoparticles carrying the polynucleotide to a magnetic force.

2. The method claim 1 comprising the steps of:
a. loading the polynucleotide molecules to be transferred into the spore on magnetic nanoparticles (MNP), and
b. adding said polynucleotide molecules loaded on said magnetic nanoparticles (DNA-MNP) to said spores, and
c. exposing the mixture to a magnetic force,
d. incubating the mixture in presence of said magnet to allow introduction of the polynucleotide molecules into said spores,
thereby introducing the polynucleotide molecules into said spores.

3. The method of claim 2, wherein the number of spores per millilitre in the incubation step is 10⁵ or higher.

4. The method of claim 1, wherein the polynucleotide loaded to the MNPs is DNA or RNA or a nucleotide analogon.

5. The method of claim 1, wherein the spores are transformed as non-germinated or germinated spores.

6. The method of claim 1, wherein the spore germination time for the spore before transformation is between 0 and 2 h.

7. A method for production of a transformed fungus comprising the steps of the method of claim 1 and further comprising selecting for fungal spores and/or fungal cells that have said polynucleotide present.

8. The method of claim 7, wherein the polynucleotide or a fragment thereof is stably integrated in to the genome of the spore DNA.

9. The method of claim 1, further comprising growing a fungus from the fungal spore.

10. A system for delivery of nucleic acids to fungal spores comprising magnetic nanoparticles loaded with nucleic acids.

11. A kit for transformation of fungal spores with polynucleotides comprising MNPs loaded with polynucleotides and means for the magnetofection of spores.

12. A composition comprising MNPs loaded with nucleic acid molecules, fungal spores and a buffer.

13. The composition of claim 12, wherein the spore density in the composition is more than 10⁵/ml for 100 ng to 500 ng polynucleotide.

14. The composition of claim 12, wherein the number of spores per millilitre in the composition is 10⁵ to 10⁷ for the transfection of an amount of DNA between 200 ng and 300 ng

15. The method of claim 1 wherein the polynucleotide is double strand or single strand ribonucleotide.

16. The method of claim 1, wherein the polynucleotide loaded to the MNPs is plasmid DNA or linear DNA.

17. The method of claim 1, wherein magnetic force is a magnet and/or a magnetic field.
